(19) United States
(12) Patent Application Publication
(10) Pub. No.: US 2025/0355018 A1
(43) Pub. Date: Nov. 20, 2025

(54) WIND VELOCITY MEASUREMENT APPARATUS AND METHOD FOR AIRCRAFT, AND GREENHOUSE GAS DETECTION APPARATUS

(71) Applicants: Hainan Institute of Zhejiang University, Sanya City (CN); Sanya Sixiang Agricultural Technology Co., Ltd., Sanya City (CN)

(72) Inventors: Yong HE, Sanya City (CN); Liwen HE, Sanya City (CN)

(21) Appl. No.: 19/028,052

(22) Filed: Jan. 17, 2025

(30) Foreign Application Priority Data
May 14, 2024 (CN) 202410591510.4

(51) Int. Cl. G01P 13/02; G01N 33/00; G01P 5/24; G01P 5/26

(52) U.S. Cl. CPC G01P 13/025; G01N 33/0027; G01P 5/245; G01P 5/26

(57) ABSTRACT

This application provides a wind velocity measurement apparatus for an aircraft. The wind velocity measurement apparatus for an aircraft includes an aircraft attitude obtaining apparatus, an ultrasonic or laser wind measurement apparatus, an attitude adjustment apparatus, an aircraft parameter obtaining apparatus, and a control apparatus. The ultrasonic or laser wind measurement apparatus is configured to measure a relative wind angle. The aircraft parameter obtaining apparatus is configured to obtain a ground velocity and a set course angle of an aircraft. The control apparatus is capable of adjusting, based on an attitude of the aircraft, an attitude of the ultrasonic or laser wind measurement apparatus via the attitude adjustment apparatus until a transmit end and a receive end of the ultrasonic or laser wind measurement apparatus are both kept horizontal. This application further provides a corresponding wind velocity measuring method for an aircraft, and a greenhouse gas detection apparatus.

### CROSS REFERENCE TO RELATED APPLICATION

This patent application claims the benefit and priority of Chinese Patent Application No. 202410591510.4, filed with the China National Intellectual Property Administration on May 14, 2024, the disclosure of which is incorporated by reference herein in its entirety as part of the present application.

### TECHNICAL FIELD

The present disclosure relates to the field of aircraft measuring technologies, and in particular, to a wind velocity measurement apparatus and method for an aircraft, and a greenhouse gas detection apparatus.

### BACKGROUND

At present, the conventional method for collecting greenhouse gas in farmland is as follows: An enrichment box is placed above the plant to seal the plant area, and after a period of time, the gas in the box is extracted and taken to the lab for analysis, so as to reduce disturbance of gas flow, that is caused by low-level wind shear in farmland, on gas collection. However, in the method of measuring greenhouse gas in farmland, gas samples need to be taken to the lab for analysis, and consequently, real-time performance of the data is poor and it is inconvenient for continuous observation of greenhouse gas in farmland. Therefore, an aircraft like an unmanned aerial vehicle can be configured to monitor greenhouse gas emission in farmland, to achieve real-time continuous observation of greenhouse gas in farmland. In the technology, a wind velocity and a wind direction have significant impact on test of greenhouse gas concentration and the like.

At present, when an ultrasonic or laser wind velocity and wind direction sensor is configured to measure a wind velocity and a wind direction, a transmit end and a receive end of the ultrasonic or laser wind velocity and wind direction sensor need to be kept horizontal. The ultrasonic or laser wind velocity and wind direction sensor is mainly disposed on a static object to measure a wind velocity and a wind direction at the static object. However, currently, there is no ultrasonic or laser wind measurement apparatus for measuring a wind velocity and a wind direction of an aircraft in a flight state.

### SUMMARY

An objective of the present disclosure is to provide a wind velocity measurement apparatus and method for an aircraft, and a greenhouse gas detection apparatus, to resolve the problem in the conventional technology, and measure a wind velocity and direction for the aircraft via an ultrasonic or laser wind measurement apparatus.

To achieve the above objective, the present disclosure provides the following technical solutions.

The present disclosure provides a wind velocity measurement apparatus for an aircraft, including an aircraft attitude obtaining apparatus, an ultrasonic or laser wind measurement apparatus, an attitude adjustment apparatus, an aircraft parameter obtaining apparatus, and a control apparatus. The aircraft attitude obtaining apparatus, the attitude adjustment apparatus, the aircraft parameter obtaining apparatus, and the control apparatus are all disposed on an aircraft. The ultrasonic or laser wind measurement apparatus is fixedly connected to an output end of the attitude adjustment apparatus. The ultrasonic or laser wind measurement apparatus is configured to measure a relative wind angle at the ultrasonic or laser wind measurement apparatus. The aircraft parameter obtaining apparatus is configured to obtain a ground velocity and a set course angle of the aircraft, and the aircraft attitude obtaining apparatus, the ultrasonic or laser wind measurement apparatus, the attitude adjustment apparatus, and the aircraft parameter obtaining apparatus are all connected to the control apparatus. The control apparatus is configured to control, based on an attitude that is of the aircraft and that is obtained via the aircraft attitude obtaining apparatus, the attitude adjustment apparatus to adjust an attitude of the ultrasonic or laser wind measurement apparatus until a transmit end and a receive end of the ultrasonic or laser wind measurement apparatus are both kept horizontal. The control apparatus is configured to obtain an absolute wind velocity at the ultrasonic or laser wind measurement apparatus based on the ground velocity of the aircraft, the relative wind angle and the set course angle of the aircraft. The control apparatus is configured to obtain an absolute wind angle at the ultrasonic or laser wind measurement apparatus based on the relative wind angle and the set course angle of the aircraft.

Preferably, the ultrasonic or laser wind measurement apparatus is disposed in front of the aircraft, and is disposed in front of a forward direction of a blade of the aircraft. A height of the ultrasonic or laser wind measurement apparatus is greater than that of the blade.

Preferably, a supporting rod is further included, where one end of the supporting rod is fixedly connected to the aircraft, and the other end of the supporting rod extends toward the front of the aircraft, and the other end of the supporting rod is fixedly connected to the attitude adjustment apparatus.

Preferably, the attitude adjustment apparatus includes a roll drive apparatus and a pitch drive apparatus. The roll drive apparatus is fixedly connected to an end of the supporting rod that is away from the aircraft. An output end of the roll drive apparatus is fixedly connected to the pitch drive apparatus. An output end of the pitch drive apparatus is fixedly connected to the ultrasonic or laser wind measurement apparatus, and the roll drive apparatus and the pitch drive apparatus are both in signal connection to the control apparatus. The control apparatus is configured to control the roll drive apparatus to rotate around a first axis and maintain the roll drive apparatus at a position, and is further configured to control the pitch drive apparatus to rotate around a second axis and maintain the pitch drive apparatus at a position. The first axis is colinear with or parallel to a longitudinal axis of the aircraft, and the second axis is parallel to a lateral axis of the aircraft.

Preferably, the aircraft parameter obtaining apparatus includes an aircraft ground velocity obtaining apparatus and an aircraft set course angle obtaining apparatus. The aircraft ground velocity obtaining apparatus and the aircraft set course angle obtaining apparatus are both disposed on the aircraft and are both connected to the control apparatus. The aircraft ground velocity obtaining apparatus is configured to obtain the ground velocity of the aircraft, and the aircraft set course angle obtaining apparatus is configured to obtain the set course angle of the aircraft.

Preferably, the relative wind angle is a wind angle that is measured by using the aircraft as a reference object; and the absolute wind velocity is a wind velocity that is measured by using ground as a reference object, and the absolute wind angle is a wind angle that is measured by using the ground as the reference object.

The present disclosure further provides a wind velocity measuring method for an aircraft based on the wind velocity measurement apparatus for an aircraft, including the following steps: obtaining, by the aircraft attitude obtaining apparatus, an attitude of the aircraft in a flight process, and controlling, by the control apparatus, the attitude adjustment apparatus to adjust an attitude of the ultrasonic or laser wind measurement apparatus based on the attitude of the aircraft, to enable both a transmit end and a receive end of the ultrasonic or laser wind measurement apparatus to keep horizontal; measuring, by the ultrasonic or laser wind measurement apparatus, a relative wind angle at the ultrasonic or laser wind measurement apparatus; and obtaining, by the aircraft parameter obtaining apparatus, a ground velocity and a set course angle of the aircraft; obtaining, by the control apparatus, an absolute wind velocity at the ultrasonic or laser wind measurement apparatus based on the ground velocity of the aircraft, the relative wind angle and the set course angle of the aircraft; and obtaining, by the control apparatus, an absolute wind angle at the ultrasonic or laser wind measurement apparatus based on the relative wind angle and the set course angle of the aircraft.

Preferably, the method further includes obtaining, by the control apparatus, the absolute wind velocity at the ultrasonic or laser wind measurement apparatus by using formula 1:

$${V}_{w} = {V}_{g} \times tan ( {θ}_{r} - {θ}_{h} ) , \qquad formula 1$$

where

V_{w} is the absolute wind velocity, V_{g} is the ground velocity of the aircraft, θᵣ is the relative wind angle measured by the ultrasonic or laser wind measurement apparatus, and θₕ is the set course angle of the aircraft.

Preferably, the method further includes: obtaining, by the control apparatus, the absolute wind angle at the ultrasonic or laser wind measurement apparatus by using formula 2:

$${θ}_{w} = {θ}_{h} + {θ}_{r} , \qquad formula 2$$

where θ_{w} is the absolute wind angle.

The present disclosure further provides a greenhouse gas detection apparatus, including the aircraft, a greenhouse gas detection body and the wind velocity measurement apparatus for an aircraft, where the greenhouse gas detection body, the aircraft attitude obtaining apparatus, the attitude adjustment apparatus, the aircraft parameter obtaining apparatus, and the control apparatus are all connected to the aircraft.

Compared with the prior art, the present disclosure has the following technical effects:

The present disclosure provides a wind velocity measurement apparatus and method for an aircraft, and a greenhouse gas detection apparatus. The wind velocity measurement apparatus for an aircraft includes an aircraft attitude obtaining apparatus, an ultrasonic or laser wind measurement apparatus, an attitude adjustment apparatus, an aircraft parameter obtaining apparatus, and a control apparatus, where the ultrasonic or laser wind measurement apparatus is configured to measure a relative wind angle at the ultrasonic or laser wind measurement apparatus; the aircraft parameter obtaining apparatus is configured to obtain a ground velocity and a set course angle of the aircraft; the control apparatus is configured to control, based on an attitude that is of the aircraft and that is obtained via the aircraft attitude obtaining apparatus, the attitude adjustment apparatus to adjust an attitude of the ultrasonic or laser wind measurement apparatus until a transmit end and a receive end of the ultrasonic or laser wind measurement apparatus are both kept horizontal; the control apparatus is configured to obtain an absolute wind velocity at the ultrasonic or laser wind measurement apparatus based on the ground velocity of the aircraft, the relative wind angle and the set course angle of the aircraft; and the control apparatus is configured to obtain an absolute wind angle at the ultrasonic or laser wind measurement apparatus based on the relative wind angle and the set course angle of the aircraft.

According to the wind velocity measurement apparatus for an aircraft, the attitude of the aircraft is obtained via the aircraft attitude obtaining apparatus, and the attitude adjustment apparatus is controlled via the control apparatus to adjust the attitude of the ultrasonic or laser wind measurement apparatus, so that the transmit end and the receive end of the ultrasonic or laser wind measurement apparatus are kept horizontal. This helps obtain the relative wind angle via the ultrasonic or laser wind measurement apparatus. The ground velocity and the set course angle of the aircraft are obtained via the aircraft parameter obtaining apparatus, to obtain the absolute wind velocity of the flight environment based on the ground velocity of the aircraft, the relative wind angle and the set course angle, and obtain the absolute wind angle at the ultrasonic or laser wind measurement apparatus based on the relative wind angle and the set course angle.

### BRIEF DESCRIPTION OF THE DRAWINGS

To describe the technical solutions in embodiments of the present disclosure or in the prior art more clearly, the accompanying drawings required for the embodiments are briefly described below. Apparently, the accompanying drawings in the following description show merely some embodiments of the present disclosure, and a person of ordinary skill in the art may still derive other accompanying drawings from these accompanying drawings without creative efforts.

FIG. 1 is a schematic diagram of a structure of a wind velocity measurement apparatus for an aircraft according Embodiment 1; and

FIG. 2 is a schematic diagram of a structure of a greenhouse gas detection apparatus according to Embodiment 2.

In the figures: 100: wind velocity measurement apparatus for an aircraft; 200: greenhouse gas detection apparatus; 1: aircraft attitude obtaining apparatus; 2: ultrasonic or laser wind measurement apparatus; 3: attitude adjustment apparatus; 301: roll drive apparatus; 302: pitch drive apparatus; 4: aircraft ground velocity obtaining apparatus; 5: aircraft set course angle obtaining apparatus; 6: control apparatus; 7: aircraft; 701: blade; 8: supporting rod; and 9: greenhouse gas detection body.

### DETAILED DESCRIPTION OF THE EMBODIMENTS

The technical solutions of the embodiments of the present disclosure are clearly and completely described below with reference to the drawings in the embodiments of the present disclosure. Apparently, the described embodiments are merely a part rather than all of the embodiments of the present disclosure. All other embodiments obtained by a person of ordinary skill in the art based on the embodiments of the present disclosure without creative efforts shall fall within the protection scope of the present disclosure.

An objective of the present disclosure is to provide a wind velocity measurement apparatus and method for an aircraft, and a greenhouse gas detection apparatus, to resolve the problem in the conventional technology, and measure a wind velocity and direction for the aircraft via an ultrasonic or laser wind measurement apparatus.

In order to make the above objective, features and advantages of the present disclosure clearer and more comprehensible, the present disclosure will be further described in detail below in combination with accompanying drawings and particular implementations.

### Embodiment 1

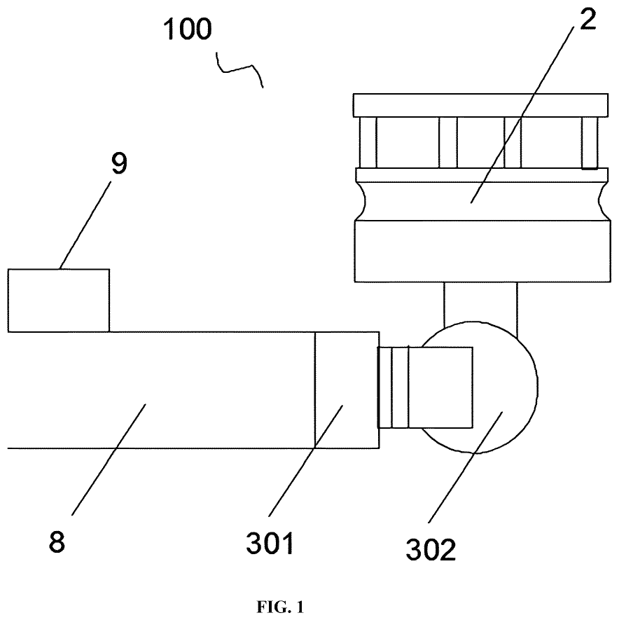

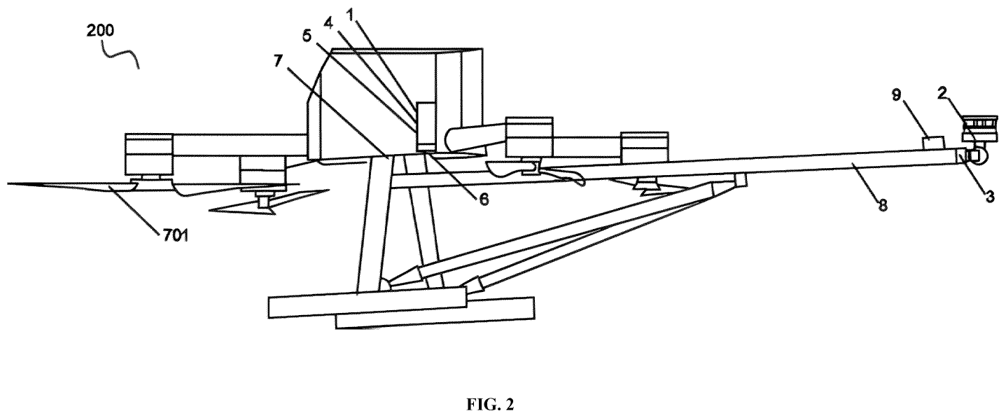

As shown in FIG. 1 and FIG. 2. This embodiment provides a wind velocity measurement apparatus 100 for an aircraft, including an aircraft attitude obtaining apparatus 1, an ultrasonic or laser wind measurement apparatus 2, an attitude adjustment apparatus 3, an aircraft parameter obtaining apparatus, and a control apparatus 6. The aircraft attitude obtaining apparatus 1, the attitude adjustment apparatus 3, the aircraft parameter obtaining apparatus, and the control apparatus 6 are all disposed on an aircraft 7. The ultrasonic or laser wind measurement apparatus 2 is fixedly connected to an output end of the attitude adjustment apparatus 3, and the ultrasonic or laser wind measurement apparatus 2 is configured to measure a relative wind angle at the ultrasonic or laser wind measurement apparatus 2. The aircraft parameter obtaining apparatus is configured to obtain a ground velocity and a set course angle of the aircraft 7. The aircraft attitude obtaining apparatus 1, the ultrasonic or laser wind measurement apparatus 2, the attitude adjustment apparatus 3, and the aircraft parameter obtaining apparatus are all connected to the control apparatus 6. The control apparatus 6 is configured to control, based on an attitude that is of the aircraft and that is obtained via the aircraft attitude obtaining apparatus 1, the attitude adjustment apparatus 3 to adjust an attitude of the ultrasonic or laser wind measurement apparatus 2 until a transmit end and a receive end of the ultrasonic or laser wind measurement apparatus 2 are both kept horizontal. The control apparatus 6 is configured to obtain an absolute wind velocity at the ultrasonic or laser wind measurement apparatus 2 based on the ground velocity of the aircraft 7, the relative wind angle and the set course angle of the aircraft 7. The control apparatus 6 is configured to obtain an absolute wind angle at the ultrasonic or laser wind measurement apparatus 2 based on the relative wind angle and the set course angle of the aircraft 7.

According to the wind velocity measurement apparatus 100 for an aircraft, the attitude of the aircraft 7 is obtained via the aircraft attitude obtaining apparatus 1, and the attitude adjustment apparatus 3 is controlled via the control apparatus 6 to adjust the attitude of the ultrasonic or laser wind measurement apparatus 2, so that the transmit end and the receive end of the ultrasonic or laser wind measurement apparatus 2 are kept horizontal. This helps obtain the relative wind angle via the ultrasonic or laser wind measurement apparatus 2. The ground velocity and the set course angle of the aircraft 7 are obtained via the aircraft parameter obtaining apparatus, to obtain the absolute wind velocity of the flight environment based on the ground velocity of the aircraft 7, the relative wind angle and the set course angle, and obtain the absolute wind angle at the ultrasonic or laser wind measurement apparatus 2 based on the relative wind angle and the set course angle.

In this embodiment, the aircraft parameter obtaining apparatus includes an aircraft ground velocity obtaining apparatus 4 and an aircraft set course angle obtaining apparatus 5. The aircraft ground velocity obtaining apparatus 4 and the aircraft set course angle obtaining apparatus 5 are both disposed on the aircraft 7. The aircraft ground velocity obtaining apparatus 4 and the aircraft set course angle obtaining apparatus 5 are both connected to the control apparatus 6. The aircraft ground velocity obtaining apparatus 4 is configured to obtain the ground velocity of the aircraft 7, and the aircraft set course angle obtaining apparatus 5 is configured to obtain a set course angle of the aircraft 7.

In a preferable implementation, the ultrasonic or laser wind measurement apparatus 2 is an ultrasonic or laser wind velocity and wind direction measurement apparatus, and is configured to measure a relative wind velocity and a relative wind angle at the ultrasonic or laser wind measurement apparatus 2. The relative wind velocity is a wind velocity that is measured by using the aircraft 7 as a reference object, and the relative wind angle is a wind angle that is measured by using the aircraft 7 as the reference object. The absolute wind velocity is a wind velocity that is measured by using ground as a reference object, and the absolute wind angle is a wind angle that is measured by using the ground as the reference object.

Specifically, the control apparatus 6, the aircraft attitude obtaining apparatus 1, the ultrasonic or laser wind measurement apparatus 2, the attitude adjustment apparatus 3, the aircraft ground velocity obtaining apparatus 4, and the aircraft set course angle obtaining apparatus 5 are all in signal connection. The aircraft attitude obtaining apparatus 1 is capable of sending an aircraft attitude signal to the control apparatus 6, and the control apparatus 6 is capable of adjusting, based on the aircraft attitude signal, an attitude of the ultrasonic or laser wind measurement apparatus 2 via the attitude adjustment apparatus 3. The ultrasonic or laser wind measurement apparatus 2 is capable of sending a relative wind angle signal to the control apparatus 6. The aircraft ground velocity obtaining apparatus 4 is capable of sending an aircraft ground velocity signal to the control apparatus 6. The aircraft set course angle obtaining apparatus 5 is capable of sending a set course angle signal to the control apparatus 6. The control apparatus 6 is configured to obtain an absolute wind velocity at the ultrasonic or laser wind measurement apparatus 2 based on the aircraft ground velocity signal, the relative wind angle signal, and the set course angle signal. The control apparatus 6 is configured to obtain an absolute wind angle at the ultrasonic or laser wind measurement apparatus 2 based on the relative wind angle signal and the set course angle signal. It should be noted that, the relative wind angle, the relative set course angle, and the absolute wind angle are all angles relative to the north, and the north represents 0°.

In this embodiment, the ultrasonic or laser wind measurement apparatus 2 is disposed in front of the aircraft 7, and is disposed in front of a forward direction of a blade 701 of the aircraft 7, and a height of the ultrasonic or laser wind measurement apparatus 2 is greater than a height of the blade 701. In this way, interference, that is of propeller slipstream and wake stream generated during flight of the aircraft 7, on airflow is reduced, and measurement accuracy of the ultrasonic or laser wind measurement apparatus 2 is ensured. In a preferable implementation, a distance between the aircraft 7 and the blade 701 is greater than 1.5 times a rotation diameter of the blade 701.

In this embodiment, a supporting rod 8 is further included. One end of the supporting rod 8 is fixedly connected to the aircraft 7, and the other end of the supporting rod 8 extends toward the front of the aircraft 7. The other end of the supporting rod 8 is fixedly connected to the attitude adjustment apparatus 3. The ultrasonic or laser wind measurement apparatus 2 is disposed away from the tail of the aircraft 7 and all blades 701 via the supporting rod 8, so that measurement accuracy of the ultrasonic or laser wind measurement apparatus 2 is ensured.

In this embodiment, the attitude adjustment apparatus 3 includes a roll drive apparatus 301 and a pitch drive apparatus 302. The roll drive apparatus 301 is fixedly connected to an end that is of the supporting rod 8 and that is away from the aircraft 7, an output end of the roll drive apparatus 301 is fixedly connected to the pitch drive apparatus 302, an output end of the pitch drive apparatus 302 is fixedly connected to the ultrasonic or laser wind measurement apparatus 2, and the roll drive apparatus 301 and the pitch drive apparatus 302 are both in signal connection to the control apparatus 6. The control apparatus 6 is configured to control the roll drive apparatus 301 to rotate around a first axis and maintain the roll drive apparatus 301 at a position. The control apparatus 6 is configured to control the pitch drive apparatus 302 to rotate around a second axis and maintain the pitch drive apparatus 302 at a position. The first axis is colinear with or parallel to a longitudinal axis of the aircraft 7, and the second axis is parallel to a lateral axis of the aircraft 7. The longitudinal axis of the aircraft 7 is an axis in a length direction (from head to tail) of the aircraft 7, and the lateral axis of the aircraft 7 is an axis in a width direction of the aircraft 7. During flight, the aircraft 7 may tilt forward or adjust a side tilt angle to resist wind and keep flight on course. In this case, relative positions between the ultrasonic or laser wind measurement apparatus 2 and the horizontal plane are changed, and a measuring result of the ultrasonic or laser wind measurement apparatus 2 is affected. When the aircraft 7 tilts forward or rolls, the control apparatus 6 controls, based on the aircraft attitude signal sent by the aircraft attitude obtaining apparatus 1, the corresponding roll drive apparatus 301 and the pitch drive apparatus 302 to rotate, so that the relative positions between the ultrasonic or laser wind measurement apparatus 2 and the horizontal plane are kept unchanged all the time. This ensures precision of the measuring result. In a preferable implementation, the roll drive apparatus 301 and the pitch drive apparatus 302 are both motors.

In a preferable implementation, the control apparatus 6 includes a first controller and a second controller that are in signal connection. The first controller is a flight control computer of the aircraft 7, the first controller is in signal connection to the aircraft attitude obtaining apparatus 1, and the second controller is in signal connection to the attitude adjustment apparatus 3. The first controller is configured to send, based on the obtained attitude of the aircraft 7, a corresponding attitude adjustment instruction for a laser wind velocity and wind direction detection apparatus to the second controller, and the second controller is configured to adjust an attitude of the laser wind velocity and wind direction detection apparatus via the attitude adjustment apparatus 3, so that the ultrasonic or laser wind measurement apparatus 2 moves in a forward-tilt or roll direction of the aircraft 7. The aircraft set course angle obtaining apparatus 5 is integrated on the flight control computer, and a preset course angle of the aircraft 7 is input via the flight control computer, to obtain the set course angle of the aircraft 7.

In a preferable implementation, after obtaining the absolute wind direction and the absolute wind velocity of the environment, the flight control computer is capable of storing the absolute wind direction, the absolute wind velocity, and a flight log for reading and use of a user.

It should be noted that, the ultrasonic or laser wind measurement apparatus 2 may be a laser wind velocity and wind direction sensor or an ultrasonic wind velocity and wind direction sensor, and the two have a same principle of measuring a wind direction and a wind velocity. For example, when an ultrasonic wind velocity and wind direction detection apparatus is configured to measure wind, a series of ultrasonic pulses are emitted by an ultrasonic emitter of the ultrasonic wind velocity and wind direction detection apparatus, and echo signal delay is caused when the ultrasonic pulses encounter particles or dust in air. A receiver of the ultrasonic wind velocity and wind direction detection apparatus is configured to obtain the echo signals, and the ultrasonic wind velocity and wind direction detection apparatus is capable of calculating a wind velocity based on a time difference between time of emitting the ultrasonic pulses and time of receiving the echo signals. The principle of measuring a wind velocity and a wind direction by the laser wind velocity and wind direction sensor and the ultrasonic wind velocity and wind direction sensor is adopted in the prior art. Details are not described herein again. The aircraft attitude obtaining apparatus 1, for example, an MPU 6050 attitude sensor, may be adopted in the prior art. Details are not described herein again. The aircraft ground velocity obtaining apparatus 4, for example, a navigation device like an airborne Doppler navigation radar, may be adopted in the prior art. Details are not described herein again.

### Embodiment 2

This embodiment provides a wind velocity measuring method for an aircraft based on the wind velocity measurement apparatus 100 for an aircraft in Embodiment 1. The wind velocity measuring method for an aircraft includes the following steps: The aircraft attitude obtaining apparatus 1 is configured to obtain an attitude of the aircraft in a flight process 7, and the control apparatus 6 is configured to control the attitude adjustment apparatus 3 to adjust an attitude of the ultrasonic or laser wind measurement apparatus 2 based on the attitude of the aircraft 7, to enable both a transmit end and a receive end of the ultrasonic or laser wind measurement apparatus 2 to keep horizontal; the ultrasonic or laser wind measurement apparatus 2 is configured to measure a relative wind angle at the ultrasonic or laser wind measurement apparatus 2; the aircraft parameter obtaining apparatus is configured to obtain a ground velocity and a set course angle of the aircraft; the control apparatus 6 is configured to obtain an absolute wind velocity at the ultrasonic or laser wind measurement apparatus 2 based on the ground velocity of the aircraft 7, the relative wind angle and the set course angle of the aircraft 7; and the control apparatus 6 is configured to obtain an absolute wind angle at the ultrasonic or laser wind measurement apparatus 2 based on the relative wind angle and the set course angle of the aircraft 7.

In this embodiment, the wind measuring method further includes: The control apparatus 6 is configured to obtain the absolute wind velocity at the ultrasonic or laser wind measurement apparatus 2 by using formula 1:

$${V}_{w} = {V}_{g} \times tan ( {θ}_{r} - {θ}_{h} ) , \qquad formula 1$$

where

V_{w} is the absolute wind velocity, V_{g} is the ground velocity of the aircraft 7, θᵣ is the relative wind angle measured by the ultrasonic or laser wind measurement apparatus 2, and θₕ is the set course angle of the aircraft 7.

In this embodiment, the wind measuring method further includes: The control apparatus 6 is configured to obtain the absolute wind angle at the ultrasonic or laser wind measurement apparatus 2 by using formula 2:

$${θ}_{w} = {θ}_{h} + {θ}_{r} , \qquad formula 2$$

where

θ_{w} is the absolute wind angle.

### Embodiment 3

As shown in FIG. 2. This embodiment provides a greenhouse gas detection apparatus 200, including the aircraft 7, a greenhouse gas detection body 9, and the wind velocity measurement apparatus 100 for an aircraft in Embodiment 1, where the greenhouse gas detection body 9, the aircraft attitude obtaining apparatus 1, the attitude adjustment apparatus 3, the aircraft parameter obtaining apparatus, and the control apparatus 6 are all connected to the aircraft 7. The wind velocity measurement apparatus 100 for an aircraft is disposed on the aircraft, so that a wind velocity and a wind direction in a flight process can be obtained, to provide data support for an absolute wind direction and an absolute wind velocity in greenhouse gas detection. For example, the absolute wind velocity and the absolute wind angle can provide data support for an emission position at which a greenhouse gas emission source is located, to obtain a diffusion path and a diffusion distance of greenhouse gas via an atmospheric diffusion module for determining the greenhouse gas emission position and performing greenhouse gas detection at the greenhouse gas emission position. This helps improve accuracy of gas concentration detection.

In a preferable implementation, the greenhouse gas detection body 9 includes a greenhouse gas detector.

Specific examples are used herein to explain the principles and embodiments of the present disclosure. The foregoing description of the embodiments is merely intended to help understand the method of the present disclosure and its core ideas; besides, various modifications may be made by those of ordinary skill in the art to specific embodiments and the scope of application in accordance with the ideas of the present disclosure. In conclusion, the content of the present specification shall not be construed as limitations to the present disclosure.

What is claimed is:

1. A wind velocity measurement apparatus for an aircraft, comprising:
an aircraft attitude obtaining apparatus;
an ultrasonic or laser wind measurement apparatus configured to measure a relative wind angle;
an attitude adjustment apparatus, the ultrasonic or laser wind measurement apparatus is fixedly connected to an output end of the attitude adjustment apparatus;
an aircraft parameter obtaining apparatus configured to obtain a ground velocity and a set course angle of the aircraft; and
a control apparatus connected to each of the aircraft attitude obtaining apparatus, the ultrasonic or laser wind measurement apparatus, the attitude adjustment apparatus, and the aircraft parameter obtaining apparatus, the control apparatus being configured:
to control, based on an attitude that is of the aircraft and that is obtained via the aircraft attitude obtaining apparatus, the attitude adjustment apparatus to adjust an attitude of the ultrasonic or laser wind measurement apparatus until a transmit end and a receive end of the ultrasonic or laser wind measurement apparatus are both kept horizontal;
to obtain an absolute wind velocity at the ultrasonic or laser wind measurement apparatus based on the ground velocity of the aircraft, the relative wind angle and the set course angle of the aircraft; and
to obtain an absolute wind angle at the ultrasonic or laser wind measurement apparatus based on the relative wind angle and the set course angle of the aircraft
wherein the aircraft attitude obtaining apparatus, the attitude adjustment apparatus, the aircraft parameter obtaining apparatus, and the control apparatus are all disposed on the aircraft.

2. The wind velocity measurement apparatus for an aircraft according to claim 1, wherein the ultrasonic or laser wind measurement apparatus is disposed in front of the aircraft, and is disposed in front of a forward direction of a blade of the aircraft, wherein a height of the ultrasonic or laser wind measurement apparatus is greater than that of the blade.

3. The wind velocity measurement apparatus for an aircraft according to claim 1, further comprising a supporting rod having one end of the supporting rod fixedly connected to the aircraft, and an other end extending toward the front of the aircraft and being fixedly connected to the attitude adjustment apparatus.

4. The wind velocity measurement apparatus for an aircraft according to claim 3, wherein the attitude adjustment apparatus comprises:
a roll drive apparatus fixedly connected to the one end of the supporting rod that is away from the aircraft and having an output end; and
a pitch drive apparatus connected to the output end of the roll drive apparatus and having an output end fixedly connected to the ultrasonic or laser wind measurement apparatus;
the roll drive apparatus and the pitch drive apparatus are both in signal connection to the control apparatus;
the control apparatus being further configured to control the roll drive apparatus to rotate around a first axis and maintain the roll drive apparatus at a position, and to control the pitch drive apparatus to rotate around a second axis and maintain the pitch drive apparatus at a position;
the first axis being colinear with or parallel to a longitudinal axis of the aircraft, and the second axis being parallel to a lateral axis of the aircraft.

5. The wind velocity measurement apparatus for an aircraft according to claim 1, wherein the aircraft parameter obtaining apparatus comprises:
an aircraft ground velocity obtaining apparatus disposed on the aircraft and connected to the control apparatus, the aircraft ground velocity obtaining apparatus being configured to obtain the ground velocity of the aircraft; and
an aircraft set course angle obtaining apparatus disposed on the aircraft and connected to the control apparatus, the aircraft set course angle obtaining apparatus being configured to obtain the set course angle of the aircraft.

6. The wind velocity measurement apparatus for an aircraft according to claim 1, wherein the relative wind angle is a wind angle that is measured by using the aircraft as a reference object, and the absolute wind velocity is a wind velocity that is measured by using ground as a reference object, and the absolute wind angle is a wind angle that is measured by using the ground as the reference object.

7. A wind velocity measuring method for an aircraft based on the wind velocity measurement apparatus for an aircraft according to claim 1, comprising:
obtaining, by the aircraft attitude obtaining apparatus, an attitude of the aircraft in a flight process, and controlling, by the control apparatus, the attitude adjustment apparatus to adjust an attitude of the ultrasonic or laser wind measurement apparatus based on the attitude of the aircraft, to enable both a transmit end and a receive end of the ultrasonic or laser wind measurement apparatus to keep horizontal;
measuring, by the ultrasonic or laser wind measurement apparatus, a relative wind angle at the ultrasonic or laser wind measurement apparatus;
obtaining, by the aircraft parameter obtaining apparatus, a ground velocity and a set course angle of the aircraft;
obtaining, by the control apparatus, an absolute wind velocity at the ultrasonic or laser wind measurement apparatus based on the ground velocity of the aircraft, the relative wind angle and the set course angle of the aircraft; and
obtaining, by the control apparatus, an absolute wind angle at the ultrasonic or laser wind measurement apparatus based on the relative wind angle and the set course angle of the aircraft.

8. The wind velocity measuring method for an aircraft according to claim 7, further comprising: obtaining, by the control apparatus, the absolute wind velocity at the ultrasonic or laser wind measurement apparatus by using formula 1:
$${V}_{w} = {V}_{g} \times tan ( {θ}_{r} - {θ}_{h} ) , \qquad ( formula 1 )$$
wherein
V_{w} is the absolute wind velocity, V_{g} is the ground velocity of the aircraft, θᵣ is the relative wind angle measured by the ultrasonic or laser wind measurement apparatus, and θₕ is the set course angle of the aircraft.

9. The wind velocity measuring method for an aircraft according to claim 7, further comprising: obtaining, by the control apparatus, the absolute wind angle at the ultrasonic or laser wind measurement apparatus by using formula 2:
$${θ}_{w} = {θ}_{h} + {θ}_{r} , \qquad ( formula 2 )$$
wherein
θ_{w} is the absolute wind angle.

10. A greenhouse gas detection apparatus, comprising the aircraft, a greenhouse gas detection body and the wind velocity measurement apparatus for an aircraft according to claim 1, wherein the greenhouse gas detection body, the aircraft attitude obtaining apparatus, the attitude adjustment apparatus, the aircraft parameter obtaining apparatus, and the control apparatus are all connected to the aircraft.

11. The wind velocity measuring method for an aircraft according to claim 7, wherein the ultrasonic or laser wind measurement apparatus is disposed in front of the aircraft, and is disposed in front of a forward direction of a blade of the aircraft; and a height of the ultrasonic or laser wind measurement apparatus is greater than that of the blade.

12. The wind velocity measuring method for an aircraft according to claim 7, further comprising a supporting rod having one end fixedly connected to the aircraft, and an other end extending toward the front of the aircraft, and being fixedly connected to the attitude adjustment apparatus.

13. The wind velocity measuring method for an aircraft according to claim 12, wherein the attitude adjustment apparatus comprises:
a roll drive apparatus fixedly connected to the one end of the supporting rod that is away from the aircraft and having an output end and a pitch drive apparatus connected to the output end of the roll drive apparatus and having an output end fixedly connected to the ultrasonic or laser wind measurement apparatus;
the roll drive apparatus and the pitch drive apparatus are both in signal connection to the control apparatus;
the control apparatus being further configured to control the roll drive apparatus to rotate around a first axis and maintain the roll drive apparatus at a position, and to control the pitch drive apparatus to rotate around a second axis and maintain the pitch drive apparatus at a position; and
the first axis being colinear with or parallel to a longitudinal axis of the aircraft, and the second axis being parallel to a lateral axis of the aircraft.

14. The wind velocity measuring method for an aircraft according to claim 7, wherein the aircraft parameter obtaining apparatus comprises:
an aircraft ground velocity obtaining apparatus disposed on the aircraft and connected to the control apparatus, the aircraft ground velocity obtaining apparatus being configured to obtain the ground velocity of the aircraft; and
an aircraft set course angle obtaining apparatus disposed on the airfact and connected to the control apparatus, the aircraft set course angle obtaining apparatus being configured to obtain the set course angle of the aircraft.

15. The wind velocity measuring method for an aircraft according to claim 7, wherein the relative wind angle is a wind angle that is measured by using the aircraft as a reference object, and the absolute wind velocity is a wind velocity that is measured by using ground as a reference object, and the absolute wind angle is a wind angle that is measured by using the ground as the reference object.

16. The greenhouse gas detection apparatus according to claim 10, wherein the ultrasonic or laser wind measurement apparatus is disposed in front of the aircraft, and is disposed in front of a forward direction of a blade of the aircraft, wherein a height of the ultrasonic or laser wind measurement apparatus is greater than that of the blade.

17. The greenhouse gas detection apparatus according to claim 10, further comprising a supporting rod having one end fixedly connected to the aircraft, and an other end extending toward the front of the aircraft, and being fixedly connected to the attitude adjustment apparatus.

18. The greenhouse gas detection apparatus according to claim 17, wherein the attitude adjustment apparatus comprises:
a roll drive apparatus fixedly connected to the one end of the supporting rod that is away from the aircraft and having an output end; and
a pitch drive apparatus connected to the output end of the roll drive apparatus and having an output end fixedly connected to the ultrasonic or laser wind measurement apparatus;
the roll drive apparatus and the pitch drive apparatus are both in signal connection to the control apparatus;
the control apparatus being further configured to control the roll drive apparatus to rotate around a first axis and maintain the roll drive apparatus at a position, and to control the pitch drive apparatus to rotate around a second axis and maintain the pitch drive apparatus at a position;
the first axis being colinear with or parallel to a longitudinal axis of the aircraft, and the second axis being parallel to a lateral axis of the aircraft.

19. The greenhouse gas detection apparatus according to claim 10, wherein the aircraft parameter obtaining apparatus comprises:
an aircraft ground velocity obtaining apparatus disposed on the aircraft and connected to the control apparatus, the aircraft ground velocity obtaining apparatus is configured to obtain the ground velocity of the aircraft; and
an aircraft set course angle obtaining apparatus disposed on the aircraft and connected to the control apparatus, the aircraft set course angle obtaining apparatus is configured to obtain the set course angle of the aircraft.

20. The greenhouse gas detection apparatus according to claim 10, wherein the relative wind angle is a wind angle that is measured by using the aircraft as a reference object, and the absolute wind velocity is a wind velocity that is measured by using ground as a reference object, and the absolute wind angle is a wind angle that is measured by using the ground as the reference object.
